(19) United States
(12) Patent Application Publication
(10) Pub. No.: US 2021/0307771 A1
(43) Pub. Date: Oct. 7, 2021

(54) ARTICULATING LUMEN WITH A GRASPING END EFFECTOR

(71) Applicant: TransEnterix Surgical, Inc., Morrisville, NC (US)

(72) Inventors: Mohan Nathan, Raleigh, NC (US); Matthew Robert Penny, Holly Springs, NC (US)

(21) Appl. No.: 16/237,430

(22) Filed: Dec. 31, 2018

Related U.S. Application Data
(60) Provisional application No. 62/612,545, filed on Dec. 31, 2017.

(51) Int. Cl. A61B 17/29; A61B 34/30; A61B 34/00

(52) U.S. Cl. CPC A61B 17/29; A61B 34/30; A61B 2217/005; A61B 2034/302; A61B 2217/007; A61B 34/71

(57) ABSTRACT

A surgical system includes a robotic manipulator, a first surgical instrument removably mountable to the robotic manipulator and maneuverable within a body cavity by the robotic manipulator. The instrument has end effector and a working channel proportioned to receive an implement therethrough. Robotic control of the first surgical and, optionally, the implement, is managed using input from a user interface.

### BACKGROUND

There are various types of surgical robotic systems on the market or under development. Some surgical robotic systems use a plurality of robotic arms. Each arm carries a surgical instrument, or the camera used to capture images from within the body for display on a monitor. See U.S. Pat. No. 9,358,682 and US 20160058513, which are incorporated herein by reference. Other surgical robotic systems use a single arm that carries a plurality of instruments and a camera that extend into the body via a single incision. See WO 2016/057989, incorporated herein by reference. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

Increasingly, medical professionals are interested in advanced instrumentation including diagnostics and tissue characterization. Often, it is desirable to hold the tissue while performing the characterization and/or diagnostic procedure. This is typically done with multiple instruments. In laparoscopy, multiple instruments equates to multiple ports. This application describes dual function instruments that may be used to hold tissue while performing some additional diagnostic or therapeutic function with respect to the tissue. Use of such instruments with surgical robotic systems is also described.

### BRIEF DESCRIPTION OF THE DRAWINGS

FIG. 1 schematically illustrates a robotic manipulator of a type used in robotic surgical procedures.

FIG. 2 illustrates a surgical device according to the present disclosure mounted onto the manipulator of FIG. 1.

FIGS. 3-7 are embodiments of instruments according to aspects of the invention described herein.

### DETAILED DESCRIPTION

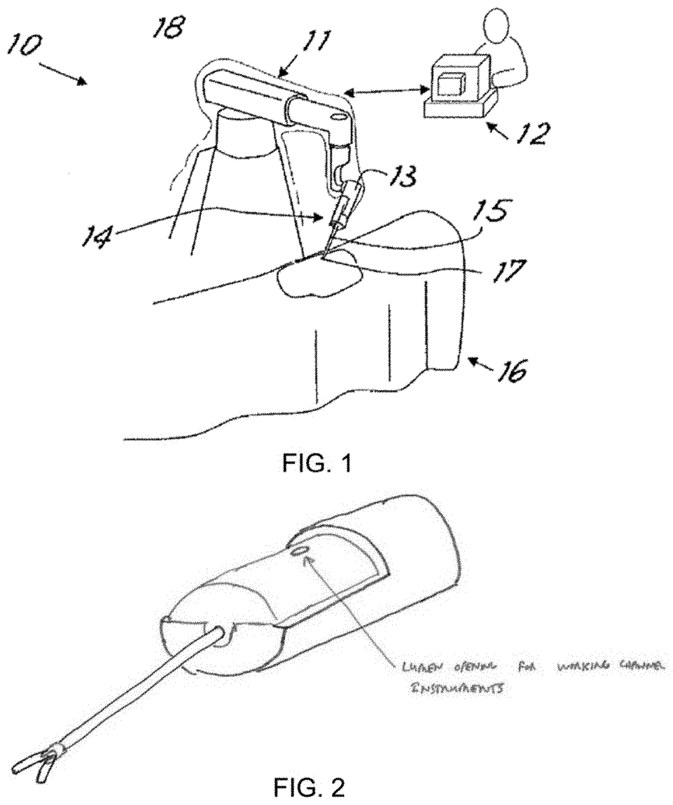

FIG. 1 shows components of a robotic surgical system 10 of the type described in U.S. Pat. No. 9,358,682 and US 20160058513. Features of the system 10 are shown to facilitate an understanding of the way in which the concepts of the present invention may be implemented, but it should be understood that the invention may be used with a variety of different surgical or industrial robotic systems and is not limited to use with system 10.

System 10 comprises at least one robot arm 11 which operates under the control of a command console 12 operated by the surgeon, as described in the Background. The robotic manipulator (or each robotic manipulator) has a terminal portion 13 designed to support and operate a surgical device assembly 14. The surgical device assembly includes a surgical instrument having shaft 15 and a distal end effector 17 positionable within a patient 16.

In this configuration, the manipulator arm receives the surgical device assembly 14 at the terminal portion 13 as shown in FIG. 2. The surgical device assembly includes a proximal housing 20 that is received by the terminal portion 13 as shown.

The end effector 17 may be one of many different types of that are used in surgery including, without limitation, end effectors 17 having one or more of the following features: jaws that open and close, section at the distal end of the shaft that bends or articulates in one or more degrees of freedom, a tip that rolls axially relative to the shaft 15, a shaft that rolls axially relative to the manipulator arm 11. The system includes instrument actuators for driving the motion of the end effector 17. These actuators, which might be motors or other types of motors (e.g. hydraulic/pneumatic), are positioned in the terminal portion 13 of the robotic manipulator, or in the housing 20 of the surgical device assembly, or some combination of the two. In the latter example, some motion of the end effector might be driven using one or more motors in the terminal portion 13, while other motion might be driven using motors in the housing 20.

During use, the robotic system controls movement of the robotic manipulator and movement of the end effector (e.g. jaw open/close, tip roll, articulating or bending, etc.) based on surgeon input received by the system via the console 12. The control signals used to generate the various types of movement depend in some cases on the geometry, length, weight, or other parameters of the surgical instrument 14.

This application describes embodiment of instruments inside of other instruments for use in surgery or medical diagnostics. In some embodiments these instruments may be rigid instruments designed for use in robotic laparoscopic procedures performed using trocars giving access into a body cavity such as the abdominal cavity. In other embodiments the internal instrument may be a flexible instrument. Embodiments described herein allow support or engagement of a tissue using an outer instrument while allowing diagnostics, characterization or even biopsy using a second instrument extending through an internal working channel of the outer instrument. The diameters of the outer instrument and the inner working channel will be selected as appropriate for the desired application. In one non-limiting example described herein, the diameter of the outer instrument shaft is 5 mm and the working channel is 2 mm. In some embodiments, the outer instrument can also articulate in one or more directions.

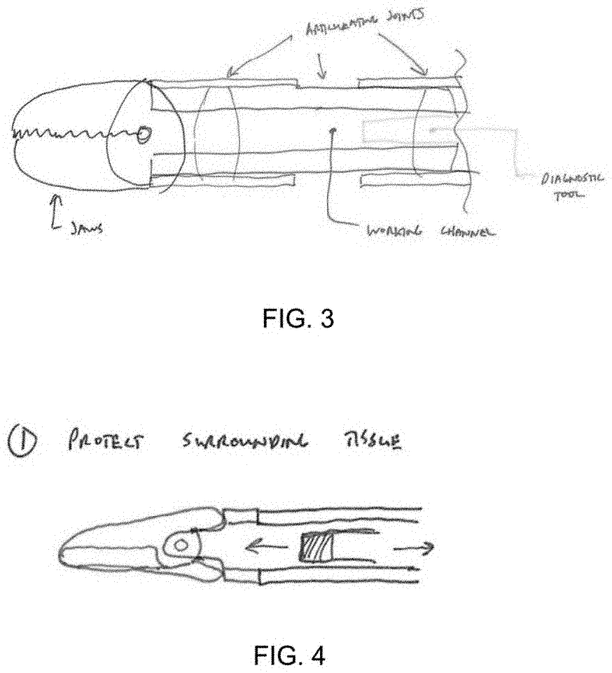

Referring to FIG. 3, outer instrument 100 has an outer shaft 102 comprised of an elongate tubular member constructed to be actively bendable in one or more directions. For example, the shaft may include reliefs to allow the tube to bend in one or more directions. An inner tubular structure 108 or shaft includes reliefs aligned with those of the outer tube to enable articulation.

The distal end of the outer shaft 102 is configured into a jaw end effector 104. The end effector may be comprised of various types and shapes, designed for different surgical interventions. Examples include, fixed-jaw needle drivers, fenestrated graspers, rat tooth graspers, shears, clamps or other atraumatic geometries. The jaw end effector may be laser cut out of the tubular member or affixed to the tubular structure in other methods.

Tendons are used to control the articulation of the outer shaft and may also be capable of controlling the open/close position of the end effector. In some cases, one component/jaw of the end effector may be fixed. In other cases, both jaws in the end effector may be pivotable.

The tendons may extend along the inner wall of the outer shaft and may be constrained between an inner tubular structure and the outer tubular shaft. In the FIG. 3 embodiment, four tendons extend from the proximal end of the tube to the jaw at the distal end. Two tendons are fixed to each jaw such that pulling on one tendon on the jaw will result in pivoting that jaw about a jaw pivot point. Pulling on both tendons will result in tube articulation at one or more relief locations.

The inner tubular structure 108 has an open working channel 110 through which other instruments or diagnostic tools 112 can be inserted to perform various functions. As an example, a fiber optic camera could be used to inspect the tissue grasped within the jaws of the outer tube for cancerous tissue. Alternatively, tools used for laser ablation may be inserted into the working channel to perform ablation on the tissue grasped within the jaws. In this case, the jaws may protect other tissue from the laser, reducing the risk of harming adjacent tissue.

In FIG. 3, the working channel 110 of the device is accessible from the external housing 20 of the instrument attached to the robotic manipulator. More specifically, an orifice or port 114 in the housing opens into a channel continuous with the working channel 110. Any device that can fit inside the inner diameter of the working channel can be fed into the exposed orifice and delivered to the operative site via the articulating instrument shaft. It should be understood that, while no attachment of the working channel implement to the instrument housing is shown, the geometry of the instrument housing could be configured to rigidly fix the working channel implement to the instrument housing such that the user may operate the implement through the surgical system interface, in addition to the instrument itself.

The instrument can have one of two primary configurations. In a first primary configuration, the outer shaft forms an open working channel through which the users could insert any second instrument or material that fits within the working channel diameter. A second primary configuration is a closed system in which the working channel is already populated within an inner instrument/working channel implement. In the second case, the working channel implement may be controlled through the same robotic manipulator as the external instrument. In the first case, the working channel implements may be controlled manually by an external user, controlled by a second robotic manipulator, or controlled by the first robotic manipulator by attaching the implement to the housing of the instrument in such a way as to enable coupling of the first robotic manipulator to the working channel implement controls.

There are a number of different applications for an instrument such as the one described above. The following examples are intended to illustrate the variety of applications, but not to be all-inclusive.

In the FIG. 4 embodiment, the outer shaft and end effector of the instrument are intended to shield or protect surrounding tissue from the intervention provided through the working channel. For example, when performing an endometrial excision, the grasper would grab and lift the target tissue away from the critical structure (in this case the ureter) and the working channel would be used to apply energy for coagulation via a laser fiber.

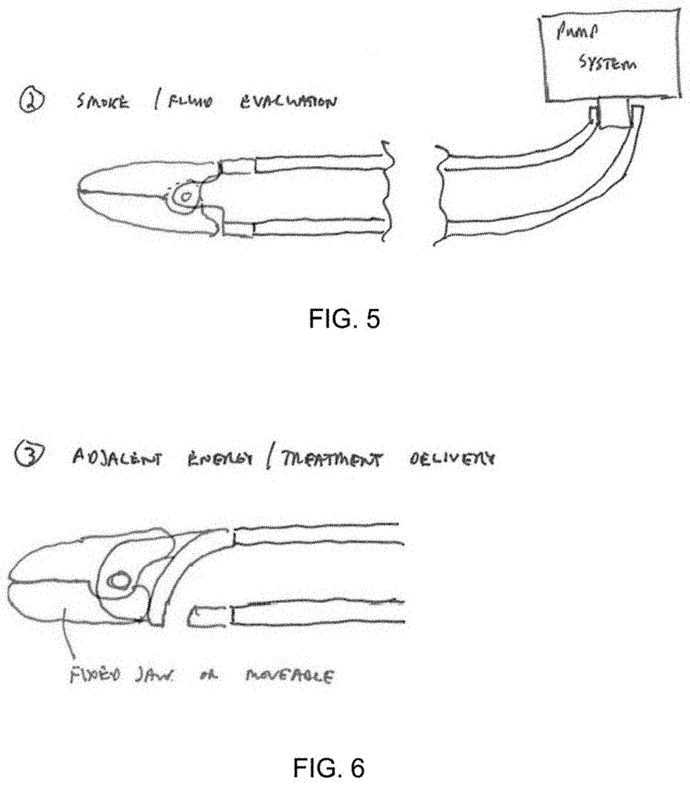

In the FIG. 5 embodiment, the outer shaft of the instrument is capable of providing a means for irrigation of a surgical site and evacuation of either fluids, tissue, or smoke. There are laparoscopic instruments currently available that provide a combination of a suction irrigator and a monopolar hook. The FIG. 4 example is configured to provide such a combination as well as combinations of suction irrigators and fenestrated graspers, and the like. In this case, the suction irrigator or other evacuation system would be attached to the instrument housing through the orifice 114. When desired, the pump system could be actuated either manually, or through the surgical system user interface.

The FIG. 6 embodiment shows a configuration in which the inner working channel enables the application of energy that would otherwise be applied through the instrument end effector. Specifically in the case of shears, the application of monopolar energy through the shear blades can lead to eschar buildup which can render the shear blades useless after only a few cycles of energy delivery. We propose using the working channel to deliver the desired energy at the tip of the instrument, but not through the end effector itself. This would allow the end effector to be used “cold”, while also allowing energy delivery through either a side port, or adjacent distal port (side port shown below).

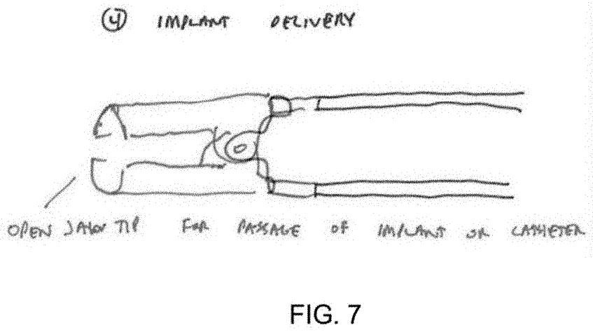

Finally, in the FIG. 7 configuration, the working channel is used to deliver a secondary component which may or may not be an implant. In the image below, the jaws are configured to be a clamp on either side of the working channel, potentially allowing the user to clamp a vessel to temporarily halt the flow of fluid through that vessel. The working channel could be used to create an opening in the vessel, between the clamps and the opening in the vessel could be used to place a valve or other device, as an example.

Other examples within the scope of this disclosure may include clip or tack delivery through the working channel or even the use of diagnostic tools such as catheters to deliver radiopaque materials to targeted tissue for imaging purposes.

The described embodiments allow the surgeon to insert various instruments or tools through a working channel of an instrument that can grasp tissue. Grasping the tissue prior to diagnostics could make the diagnostic procedures more accurate, by holding the tissue still during the characterization. It may also protect adjacent tissue from laser ablation through tissue isolation within the instrument jaws.

All patents and applications referred to herein, including for purposes of priority, are incorporated herein by reference.

What is claimed is:

1. A surgical system comprising:
a robotic manipulator;
a first surgical instrument removably mountable to the robotic manipulator and maneuverable within a body cavity by the robotic manipulator, said instrument having an end effector and a working channel proportioned to receive an implement therethrough, and wherein the control of at least the first surgical instrument is managed through a user interface.

2. The surgical instrument of claim 1, wherein the first instrument is articulatable in at least one degree of freedom.

3. The system of claim 2, wherein the first instrument includes at least one jaw moveable between first and second positions, and a tendon system actuatable by the robotic system to move the jaw between the first and second positions and to articulate the instrument.

4. The system of claim 1, wherein the first surgical instrument has a docked position in which the first surgical instrument is mounted to the robotic manipulator, and an undocked position in which the first surgical instrument is at least partially separated from the robotic manipulator, wherein the working channel is only accessible for insertion of the implement when the first instrument is in the undocked position.

5. The system of claim 1, wherein the first surgical instrument has a docked position in which the first surgical instrument is mounted to the robotic manipulator, and an undocked position in which the first surgical instrument is at least partially separated from the robotic manipulator, wherein the working channel is accessible for insertion of the implement when the first instrument is in the undocked position and in the docked position.

6. The system of claim 5, where the first surgical instrument has an external surface and wherein the implement is dockable to the external surface of the instrument, following insertion of the implement through the working channel.

7. The system of claim 1, wherein the implement has a docked in which the implement extends through the working channel and in which at least one function or degree of freedom of the implement is controllable by the robotic system via the robotic user interface.

8. The system of claim 1, where the implement manually controllable to perform an action within a body cavity when the implement is disposed in the working channel.

9. The system of claim 1, further including a second robotic manipulator, where the implement is robotically controlled by the second robotic manipulator when the implement is within the working channel.

10. The system of claim 1 where the instrument end effector is configured to protect surrounding tissue from an intervention performed using the implement.

11. The system of claim 10, wherein the instrument end effector electrically, thermally or optically isolates the surrounding tissue.

12. The system of claim 1, where the working channel is connectable to a fluid or suction source for use in delivering fluid or gas or evacuating surgical materials, fluids or devices from a working area within a body cavity.

13. The system of claim 1, wherein the working channel has a distal end positioned proximal to the distal end of the end effector.

14. The system of claim 13, wherein the distal end of the working channel is coupled with an opening in a sidewall of the instrument shaft.

15. The system of claim 14, where the instrument is extendable from the working channel via the opening in the sidewall.
